(19) United States
(12) Patent Application Publication
(10) Pub. No.: US 2017/0239081 A1
(43) Pub. Date: Aug. 24, 2017

(54) HEATED PACKS

(71) Applicant: Schawbel Technologies LLC, Burlington, MA (US)

(72) Inventors: Veronica M. Zsolcsak, Newburyport, MA (US); Micha Eizen, Lake Forest, CA (US); Thomas John William Bayes, Rothwell, Northamptonshire (GB); Ian Nicholson Whitehead, Concord, MA (US)

(73) Assignee: Schawbel Technologies LLC, Burlington, MA (US)

(21) Appl. No.: 15/506,500

(22) PCT Filed: Jul. 1, 2015
(86) PCT No.: PCT/US2015/038801

§ 371 Date: Feb. 24, 2017

Related U.S. Application Data
(60) Provisional application No. 62/043,358, filed on Aug. 28, 2014.
(60) Provisional application No. 62/091,057, filed on Dec. 12, 2014.

(51) Int. Cl. A61F 7/02; F28D 20/00; A61F 7/00; C09K 5/06

(52) U.S. Cl. CPC A61F 7/02; C09K 5/063; F28D 20/0034; F28D 20/0056; A61F 7/007; A61F 2007/0071; A61F 2007/0078; A61F 2007/0292

(57) ABSTRACT

The present invention is directed to personal heating devices, known as heated packs. Heated packs of the invention include a frame and a heating assembly disposed within the frame. The heating assembly may include a heating panel, an intelligent circuit, and a battery. In certain embodiments, one or more sides of the frame include a material configured to absorb heat or release heat in response to the energy outputted by the heating assembly. In further embodiments, the intelligent circuit is configured to change the temperature of the heating pack based a sensory input received by a user.

### RELATED APPLICATIONS

This application claims the benefit of and priority to U.S. Provisional No. 62/091,057, filed Dec. 12, 2014 and U.S. Provisional No. 62/043,358, filed Aug. 28, 2014, which are incorporated by reference herein.

### TECHNICAL FIELD

The present invention relates to personal heating devices, namely heated packs.

### BACKGROUND

Several occupations require employees to endure harsh weather conditions during the winter months. To name a few, soldiers, construction workers, agricultural workers, and law enforcement officers must routinely spend several hours outdoors despite cold, snowy or icy conditions. Others happily brave cold weather in order to enjoy activities such as skiing, hiking, snowshoeing, and sledding. Further, many must bear freezing temperatures after a snowstorm to shovel their car out and to clear accumulated snow from their driveway and/or sidewalk.

Regardless of whether one is exposed to cold weather conditions for work, fun, or chores, most accessorize with coats, boots, hats, and gloves to make the cold weather bearable. In addition to those accessories, which simply retain body heat, heated packs have recently been introduced in order to produce heat and delivery that heat directly to a user. Heated packs are generally small compact units that emit heat and are easy to carry (either hand-held or in clothing). There are two main types of heated packs. Some include an internal heater that is electrically-powered or battery-powered. Others are heated by an external power source (e.g. microwave) prior to use and that heat continually dissipates during use.

Prior art heated packs are associated with several deficiencies. These deficiencies include, for example, the inability to achieve a desired temperature and the ability to maintain the desired temperature over a period of time. Without proper temperature control, prior art heated packs are prone to overheating, which potentially leads to injury, and often prematurely lose their heat (either from dissipation or limited battery power).

### SUMMARY

The present invention provides heated packs with improved functionality and battery retention. Those features are achieved by the inclusion of an intelligent circuit, incorporation of a phase-changing material, or both. The intelligent circuit includes a feedback loop that adjusts the temperature (and thus demand on the battery) based on user commands or temperature sensors. With the feedback loop, heated packs of the invention can prolong the life of the battery and maintain heating capabilities for 6 hours or more. Additionally, one or more sides of the heated pack may include a material capable of storing and releasing heat, such as a phase changing material. Phase changing materials emit energy after the battery is depleted and assist in maintaining a constant temperature.

Personal heating devices of the invention generally include a frame and a heating assembly disposed within the frame. The frame includes a base portion and defines a recess, and the heating assembly is disposed within the recess. In certain embodiments, the frame is formed from a polymeric material, which is preferably rigid/hard to protect the heating assembly. The frame may also include a cover layer that covers the recess. The cover layer may be formed from a temperature regulating material that transitions between storing and releasing heat in response to energy outputted by the heating assembly.

Heating assemblies, according to aspects of the invention, include a battery, a control circuit, and a heating panel. The control circuit operably couples the battery to the heating panel. The control circuit provides control transfer of energy from the battery to the heating panel. In certain embodiments, the control circuit has a feedback loop configured to adjust the temperature output of the heating assembly based on a sensory input. The sensory input may be an application of pressure or a change in temperature. Particularly, the feedback loop of the circuit involves maintaining an idle temperature of the heating panel, receiving a sensory input, changing the temperature of the heating panel based on the sensory input, and returning to the idle temperature after a pre-determined period of time.

### BRIEF DESCRIPTION OF DRAWINGS

FIG. 1 depicts a heated pack of the invention.

FIG. 1A depicts a frame of the heated pack of FIG. 1.

FIG. 2 illustrates a heated pack with a woven PCM fabric as the cover layer 20.

FIGS. 3A-3B provides an internal view of the heated pack of FIG. 1.

FIG. 4 shows the unassembled elements of a heating assembly.

FIG. 5A shows a heating panel placed in a buffer structure.

FIG. 5B shows a battery coupled to the circuit in the absence of a buffer structure.

FIG. 6 provides a flow chart for controlling temperature of the heated pack.

FIG. 7 illustrates an additional embodiment of a heated panel.

### DESCRIPTION

The present invention provides heated packs with improved battery retention and functionality. Those features are achieved by the inclusion of an intelligent circuit, incorporation of a phase-changing material, or both. The intelligent circuit includes a feedback loop that is adjusts the temperature (and thus demand on the battery) based on both user commands and temperature sensors. With the intelligent feedback loop, heated packs of the invention are able to prolong the battery life and maintain heating capabilities for 6 hours or more. Additionally, one or more sides of the heated pack may include a material capable of storing and releasing heat, such as a phase changing material. Phase changing materials emit energy after the battery is depleted and assist in maintaining a constant temperature. The above-referenced features of heated packs of the invention are described in more detail hereinafter with reference to the figures.

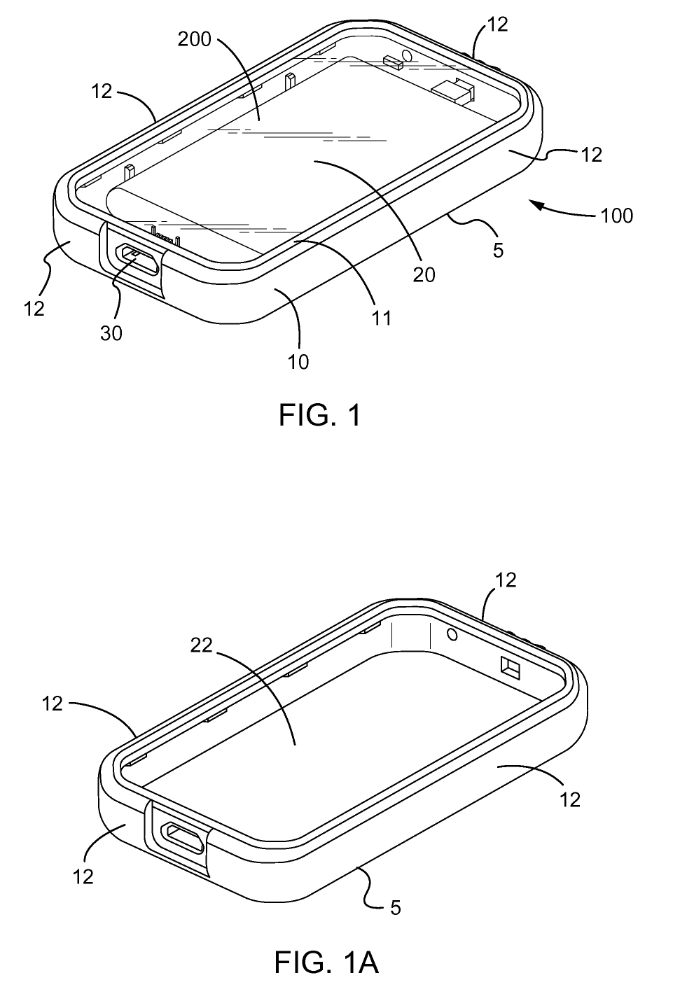

FIG. 1 illustrates an exemplary heated pack 100 of the invention. The heated pack 100 includes a frame 10 and a heating assembly disposed within a recess 22 of the frame 10 (see FIG. 1A). The frame includes any body, housing, case, or container that encompasses the heating assembly and protects the heating assembly from the environment. The frame 10 is typically rectangular in shape but other shapes are suitable, such as squares, ovals, etc. The frame 10 defines a recess 22 and includes the following elements: a base 5, one or more raised sides 12 (typically four) and optionally a cover layer 20. Those elements may be formed from the same material or from different materials. For example, one side may be formed from one material and one side may be formed from another material. Additionally, one or more of the frame elements may be formed as a unitary structure or may be coupled together. The heating assembly, which is described in more detail hereinafter, is contained within the recess 22.

In certain embodiments, the base 5 and sides 12 are formed or coupled together to define the recess 22 (See FIG. 1A). The cover layer 20 may then be used to cover the recess 22, thereby containing the heating assembly therein. The cover layer 20 may be coupled to the sides of the frame 10 via a hinge, and then closed to cover the recess 22. Alternatively, the cover layer 20 may be a separate item that is placed onto and coupled to or contained within the frame 10. In some embodiments, the base 5 and sides 12 are formed from a hard polymeric material and the cover layer is formed from a different material configured to transfer heat from the heated pack 100. The hard polymeric material of the base 5 and sides 12 protects the heating assembly, while the cover layer promotes transfer of the energy from the heating assembly to the user. In certain embodiments, a soft polymeric casing may be placed over the base 5 and sides 12. The soft polymeric casing further protects the heating assembly from impact damage (e.g. if the heated pack 100 is dropped) and/or water damage.

In certain embodiments, the cover layer is contained within the frame and directly coupled to one or more components disposed within the recess. For example, the cover layer may be coupled to a heated panel within the frame and then contained within the frame via one or more ledges or lips 11 of the frame 10 or a casing covering the frame 10.

The dimensions of the heated pack 100 may be chosen for the particular use. For example, heated pack 100 may be designed to fit in a jacket pocket may be larger than those designed to fit in gloves. For smaller heated pack 100, the dimension of the frame 10 or the entire heated pack may be 74.5 mm×41 mm×11.5 mm. For larger heated pack 100, the dimension of the frame 10 or entire heated pack may be 103 mm×71 mm×11.5 mm. The length of the heated pack may range, for example, from 25 mm to 300 mm. The width of the heated pack may range from, for example 25 mm to 300 mm. The height of the heated pack may range, for example, from 5 mm to 25 mm. The weight of the heated pack is preferably such that the heated pack can easily be carried. The heated pack may be designed to weigh, for example, 1, 2, 3, 4, 5, 6, 7, 8, 9, 10, 15, 20, 25, 30 ounces. It is understood that the above ranges are examples, and the heated pack may have dimensions and weights that vary from the cited ranges.

Suitable materials that form the frame (e.g., base 10, sides 12, or cover layer 12) are described hereinafter. The base and sides of the frame are typically formed from a plastic, polymer, or polymeric blend, although synthetic fabrics and natural fabrics may also be used. For example, the material of the frame 10 may include Polyethylene terephthalate (PET), Polyethylene (PE), High-density polyethylene (HDPE), Polyvinyl chloride (PVC), Polyvinylidene chloride (PVDC), Low-density polyethylene (LDPE), Polypropylene (PP), Polystyrene (PS), High impact polystyrene (HIPS), and combinations thereof. The material chosen for the base 10 and sides 12 is preferably lightweight, thin, and water-resistant.

The cover layer 20 is typically formed from synthetic fabrics or natural fabrics, but may also include a plastic, polymer, of polymeric blend. In certain embodiments, the cover layer may be formed from a combination of the aforementioned elements. For example, the cover layer may include a thin polymer layer and a fabric layer. Ideally, the cover layer 20 is formed from one or more temperature regulating materials. In certain embodiments, that material is a phase-changing material. It is understood that the base 10 or the sides 12 may also include a PCM material.

In general, a phase change material can be any substance (or any mixture of substances) that has the capability of absorbing or releasing thermal energy to regulate, reduce, or eliminate heat flow within a temperature stabilizing range. The temperature stabilizing range can include a particular transition temperature or a particular range of transition temperatures. When used in conjunction with heated pack 100, the PCM(s) transition between storing heat and releasing heat in response to energy outputted by the heating assembly.

A phase-change material (PCM) is a substance that melts and solidifies at a certain temperature. Heat is absorbed or released when the material changes from solid to liquid and vice versa; thus, PCMs are classified as latent heat storage (LHS) units. PCMs latent heat storage can be achieved through solid-solid, solid-liquid, solid-gas and liquid-gas phase change. Preferably, the PCM material used in the heated packs transitions from solid to liquid phase. Initially, the solid-liquid PCMs behave like sensible heat storage (SHS) materials; their temperature rises as they absorb heat. When PCMs reach the temperature at which they change phase (their melting temperature) they absorb large amounts of heat at an almost constant temperature. The PCM continues to absorb heat without a significant rise in temperature until all the material is transformed to the liquid phase. When the ambient temperature around a liquid material falls, the PCM solidifies, releasing its stored latent heat. A large number of PCMs are available in any required temperature range from −5 up to 190° C., in which the human comfort range is between 20-30° C. They may store 5 to 14 times more heat per unit volume than conventional storage materials such as water, masonry or rock.

PCM materials may be formed from organic substances, inorganic substances or polymeric substances. Examples of organic or inorganic phase change materials include hydrocarbons (e.g., straight-chain alkanes or paraffinic hydrocarbons, branched-chain alkanes, unsaturated hydrocarbons, halogenated hydrocarbons, and alicyclic hydrocarbons), hydrated salts (e.g., calcium chloride hexahydrate, calcium bromide hexahydrate, magnesium nitrate hexahydrate, lithium nitrate trihydrate, potassium fluoride tetrahydrate, ammonium alum, magnesium chloride hexahydrate, sodium carbonate decahydrate, disodium phosphate dodecahydrate, sodium sulfate decahydrate, and sodium acetate trihydrate), waxes, oils, water, fatty acids, fatty acid esters, dibasic acids, dibasic esters, 1-halides, primary alcohols, secondary alcohols, tertiary alcohols, aromatic compounds, clathrates, semi-clathrates, gas clathrates, anhydrides (e.g., stearic anhydride), ethylene carbonate, polyhydric alcohols (e.g., 2,2-dimethyl-1,3-propanediol, 2-hydroxymethyl-2-methyl-1,3-propanediol, ethylene glycol, polyethylene glycol, pentaerythritol, dipentaerythritol, pentaglycerine, tetramethylol ethane, neopentyl glycol, tetramethylol propane, 2-amino-2-methyl-1,3-propanediol, monoaminopentaerythritol, diaminopentaerythritol, and tris(hydroxymethyl)acetic acid), polymers (e.g., polyethylene, polyethylene glycol, polyethylene oxide, polypropylene, polypropylene glycol, polytetramethylene glycol, polypropylene malonate, polyneopentyl glycol sebacate, polypentane glutarate, polyvinyl myristate, polyvinyl stearate, polyvinyl laurate, polyhexadecyl methacrylate, polyoctadecyl methacrylate, polyesters produced by polycondensation of glycols (or their derivatives) with diacids (or their derivatives), and copolymers, such as polyacrylate or poly(meth)acrylate with alkyl hydrocarbon side chain or with polyethylene glycol side chain and copolymers including polyethylene, polyethylene glycol, polyethylene oxide, polypropylene, polypropylene glycol, or polytetramethylene glycol), metals, and mixtures thereof.

Polymeric phase change materials can be formed by polymerizing octadecyl methacrylate, which can be formed by esterification of octadecyl alcohol with methacrylic acid. Also, polymeric phase change materials can be formed by polymerizing a polymer (or a mixture of polymers). For example, poly-(polyethylene glycol) methacrylate, poly-(polyethylene glycol) acrylate, poly-(polytetramethylene glycol) methacrylate, and poly-(polytetramethylene glycol) acrylate can be formed by polymerizing polyethylene glycol methacrylate, polyethylene glycol acrylate, polytetramethylene glycol methacrylate, and polytetramethylene glycol acrylate, respectively. In this example, the monomer units can be formed by esterification of polyethylene glycol (or polytetramethylene glycol) with methacrylic acid (or acrylic acid). It is contemplated that polyglycols can be esterified with allyl alcohol or trans-esterified with vinyl acetate to form polyglycol vinyl ethers, which in turn can be polymerized to form poly-(polyglycol) vinyl ethers. In a similar manner, it is contemplated that polymeric phase change materials can be formed from homologues of polyglycols, such as, for example, ester or ether endcapped polyethylene glycols and polytetramethylene glycols.

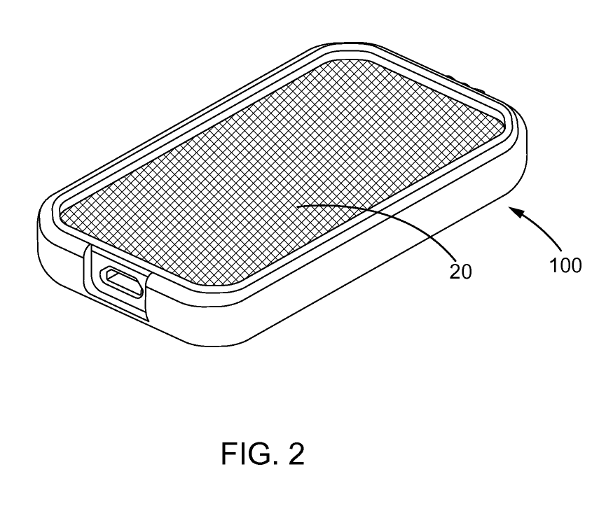

Due to the transitioning nature of PCMs (solid-liquid), it is desirable to contain the PCM materials. The phase-change material may be encapsulated (e.g. in a microcapsule) or may be contained within a fiber. Microcapsules can be formed as shells enclosing a phase change material, and can include individual microcapsules formed in various regular or irregular shapes (e.g., spherical, spheroidal, ellipsoidal, and so forth) and sizes. Microcapsules containing PCM materials can be used in a variety of manners. For example, PCM microcapsules may be used to coat a polymeric or fabric layer. Alternatively, PCM microcapsules can be dispersed throughout a polymeric or fabric layer. In other embodiments, PCM can be directly incorporated into a fibers used to make fabrics. The PCM may be located, within the core of a cellulosic fiber. In certain embodiments, fibers with PCMs incorporated therein include acrylic, viscose, and polyester fibers. FIG. 2 illustrates the heated pack 100 with a woven PCM fabric as the cover layer 20.

The type of PCM material chosen may be dependent on the desired temperature range of the heated pack 100. A transition temperature of a phase change material typically correlates with a desired temperature or a desired range of temperatures that can be maintained by the phase change material. For example, a phase change material may be selected because it has a transition temperature near the desired energy outputs (e.g. low, medium, high) of the heated pack 100. In some instances, a phase change material can have a transition temperature in the range of about −5° C. to about 125° C., such as from about 0° C. to about 100° C., from about 0° C. to about 50° C., from about 15° C. to about 45° C., from about 22° C. to about 40° C., or from about 22° C. to about 28° C.

PCMs are described in more detail in U.S. Pat. Nos. 6,855,422; 7,241,497; 7,160,612; 7,666,502; 7,666,500; 6,793,856; 7,563,398; 7,135,424; 7,244,497; 7,579,078; and 7,790,283. Also, the following references discuss phase-changing materials in more detail: Kenisarin, M; Mahkamov, K (2007). “Solar energy storage using phase change materials”. Renewable and Sustainable Energy Reviews 11 (9): 1913-1965; Sharma, Atul; Tyagi, V. V.; and Chen, C. R.; Buddhi, D. (2009). “Review on thermal energy storage with phase change materials and applications”. Renewable and Sustainable Energy Reviews 13 (2): 318-345.

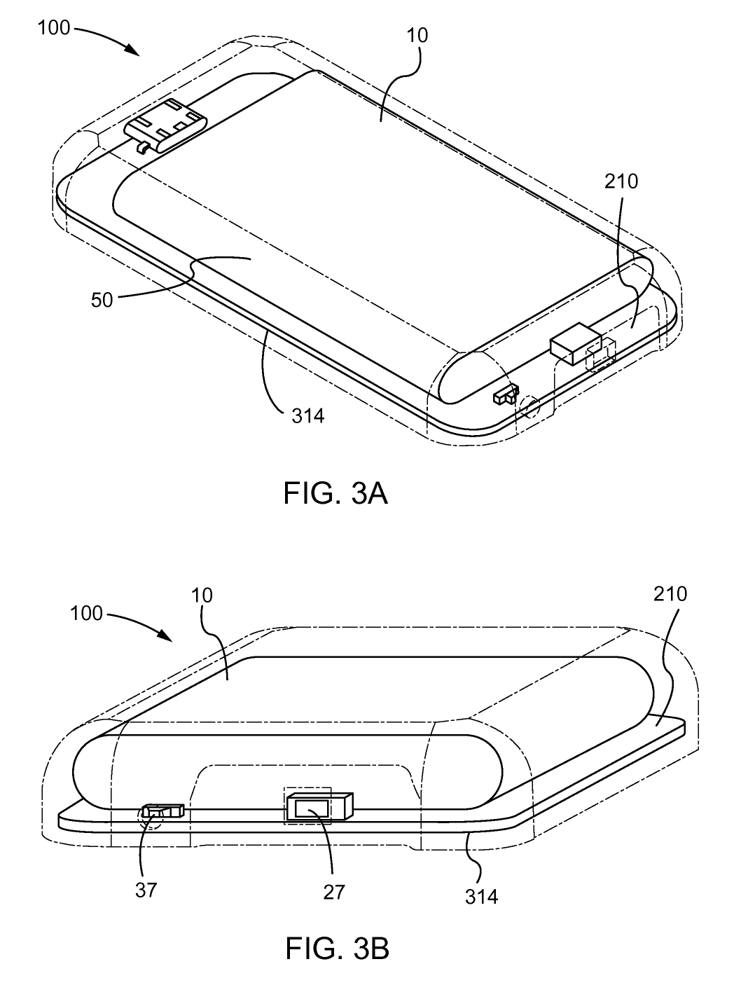

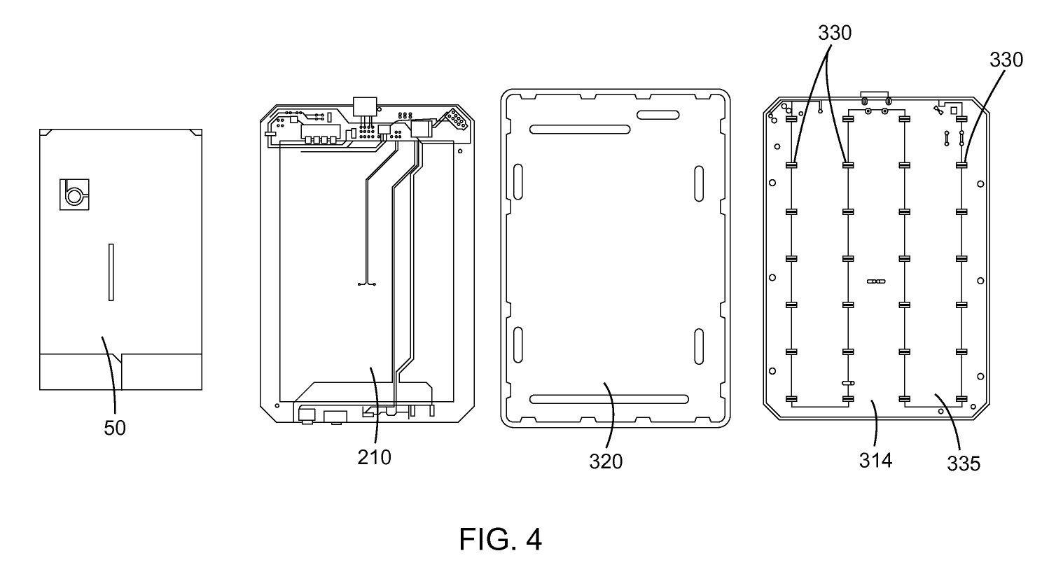

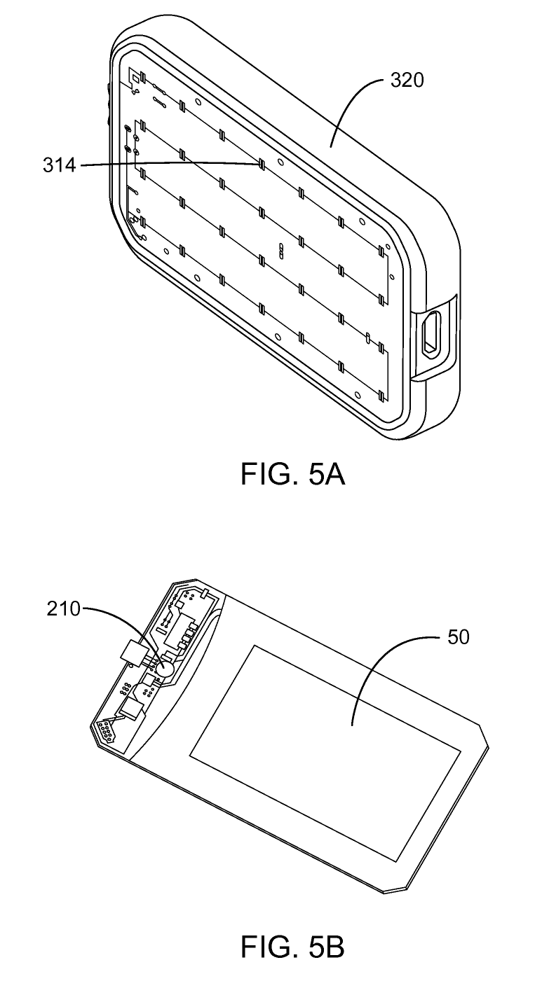

Referring back to the structure of the heated packs, a heated pack 100 includes a heating assembly 200 disposed within the recess 22 of the frame 10. FIGS. 3A and 3B illustrate show the heating assembly 200 through a transparent frame 10. The heating assembly 200 includes a battery 50, a heating panel 314, and an intelligent circuit 210. As shown in FIGS. 3A and 3B, the heating panel 314 and intelligent circuit 210 are formed on a substrate, in which the intelligent circuit is on one side of the substrate, and the heating panel is on another side of the substrate. Alternatively, the heating panel 314 and intelligent circuit 210 may be formed as separate units, e.g., on separate substrates. It is understood that the heating assembly may include one or more batteries 50, one or more intelligent circuits, and one or more heating panels 314. The circuit 210 is coupled to the heating panel 314 and the battery 50. FIG. 4 shows elements of the heating assembly when unassembled. When assembled and placed within the recess, the heating pad 314, battery 50, and circuit 210 are ideally stacked to fit within the compact frame 10 of the heating pack. Preferably, the heating assembly is placed in the recess 22 of the frame 10 such that the heating panel 314 is adjacent to the cover layer 20. This placement maximizes energy transfer from the heating panel 314 through the cover layer 20 and to a user. The intelligent circuit 210 is usually placed between the heating panel 314 and the battery 50. FIG. 5A shows the heating panel 314 is placed inside the recess of the frame 10 such that the heating panel forms a surface of the frame 10. As discussed above, the heating panel 314 may be then be covered or enclosed by a cover layer 20.

In some instances, one or more buffer structures 320 can be placed around or between any of the heating assembly elements. The buffer structure 320 can prevent or minimize undesirable overheating of the heating assembly elements. The buffer structure 320 may include recesses to at least partially contain the separate elements of the heating assembly. FIG. 5B shows the circuit 210 is coupled to the battery 50 without a buffer structure 320.

The sizes of the heating assembly elements may vary based on the desired size of the heated pack 100. For example, the heating panel 314, the circuit 210, and the battery 50 may increase in size as the heated pack 100 increases in size. The heating panel 314, battery 50, and circuit 210 may be any desirable shape. Preferably, one or more of the heating assembly elements are substantially flat and designed to fit within the frame 10 of the heated pack 100.

Suitable batteries for the heated pack include, for example, lithium-ion batteries. The lithium-ion battery may be, for example, 3.7V battery with a charging limit of 4.2V.

According to certain embodiments, the heated panel 314 is a substrate 335 with a plurality of resistors 330 in electrical communication with each other. The substrate may be formed from a flexible or a rigid material. Suitable materials for the substrate 335 include metals, such as copper, aluminum, gold, brass, silver. In some embodiments, the substrate 335 may be at least partially surrounded by an insulating film or laminate. The resisters 330 may be positioned on the substrate 335 in a random or an organized manner. The resistors 330 effectuate heat transfer across the substrate 335 from energy received from the battery 50 via the intelligent circuit 210. As shown in FIG. 4, the resistors 330 are spread-out on the substrate 335 in a plurality of rows. By spreading the resistors 330 across the substrate 335, more uniform heat transfer can be achieved. The number of resistors 330 used may vary depending on the size of the heated panel 314 and/or heated pack 100. Smaller heated packs (with dimensions described above) may include, for example, 12 resistors. Larger heated packs (with dimensions described above) may include, for example, 28 resistors. As an alternative to resistors 330, a conductive wire can be mapped onto the substrate 335 and used to dissipate heat across the substrate 335. The conductive wire is preferably placed on the substrate 335 in a serpentine pattern 318.

In certain embodiments, the substrate 335 includes both the heated panel 314 and the circuit 210. For example, one side of the substrate 335 may include the elements of the heated panel 314 and the other side of the substrate 335 may include the elements of the circuity 210.

According to certain aspects, the cover layer 20 (see FIG. 1) is coupled directly to the heating assembly 200 and placed within the frame 20 of the heat pack. The cover layer 20 may be coupled to a component of the heating assembly via an adhesive. The cover layer 20 may directly couple to the circuit 210, heated panel 314, or substrate of both the circuit and heated panel. The cover layer 20 coupled to the heating assembly 200 may also be coupled to the frame 12 or contained within the frame 12 via a lip or ledge 11 of the frame 12. In such manner, the cover layer 20 encloses the recess 22 of the frame 12.

The heated panel 314, battery 50, and circuit 210 may be coupled via one or more electrical wires. Preferably, the circuit 210 effectuates energy transfer from the battery 50 to the heating panel 314. The battery 50 may be electrically connected to the circuit 210, which in turn in electrically connected to the heating panel 314. For example, one or more first cables may have a first end that is soldered or otherwise electrically connected to battery 50 and a second end that is connected to the heater pad 314; and one or more second cables may have a first end that is soldered or otherwise electrically connected to circuit 210 and a second end that is connected to the heater pad 314. FIG. 5B illustrates the battery 50 electrically coupled to the circuit board 210.

The circuit 210 may include a processor and memory that allow the circuit to execute one or more commands based on user inputs and sensory inputs (e.g. temperature change or pressure change (i.e. sensitive to touch). The circuit 210 is configured to adjust the level of energy transferred from the battery 50 to the heater panel 314. For example, the circuit 210 may be programmed to provide certain heating levels, e.g., low, medium, and high. In some embodiments, the circuit 210 may be operably associated with a temperature sensor, and the circuit 210 delivers energy to maintain a certain threshold temperature level (such as body temperature) in response to readings transmitted from the temperature sensor. In certain embodiments, the circuit 210 may be controlled via buttons or switches located on the heated pack 100. FIG. 3B depicts a button 37 on the heated pack that may be used to turn the heated pack on/off and adjust its temperature setting. Additionally, the circuit may be controlled by a remote control. In remote control embodiments, the circuit 210 includes a receiver that receives signal from a remote, decodes the signal, and then the circuit 210 executes the operation based on the signal.

Remote control technology is generally known, and relies on sending a signal, such as light, Bluetooth (i.e. ultra-high frequency waves), and radiofrequency, to operate a device or circuit. Dominant remote control technologies rely on either infrared or radiofrequency transmissions. A radiofrequency remote transmits radio waves that correspond to the binary command for the button you're pushing. As applicable to the heated pack 100, the command may include, for example, high heat, low heat, medium heat, on, or off. A radio receiver on the controlled device (e.g. circuit 210 of heating assembly 220) receives the signal and decodes it. The receiver then transmits the decoded signal to the circuitry, and the circuitry executes the command. The above-described concepts for radiofrequency remote controls are applicable for light and Bluetooth remote controls.

According to certain aspects, one or more of the heating assembly elements (i.e. battery 50, circuit 210, and heater pad 314) are partially or completely coated or sealed with sealants, coatings, or other water proofing substances. Water proofing allows the heated pack to maintain function/operation when exposed to moisture and water.

In certain embodiments, the circuit 210 directs energy from the battery 50 to the heating panel 314 based on one or more inputs. The inputs may include user command inputs or sensory inputs. The user command inputs are those directly initiated by a user, for example, by pressing a button associated with a command (on/off, high temp, low temp, etc.). The button may be on the heated pack 100 or on a remote control that is in communication with the heated pack 100. Sensory inputs include a change in temperature or a change in pressure. A change in temperature may include a drop in the heat packs internal temperature due to changing environmental conditions. For example, a heat pack placed directly next to a person's hand (which is body temperature) will perceive a different temperature from a heat pack placed in a jacket pocket (which is more susceptible to mirror the temperature of an outdoor environment). A change in pressure may involve sensing the pressure caused when a user presses against the heat pack. The threshold may be created that distinguishes intended pressure inputs and unintended pressure input. The command or sensory inputs may cause the circuity 210 to adjust the temperature of the heat pack or adjust the amount of energy delivered from the battery 50 to maintain a certain temperature.

In certain embodiments, the circuity may include a feedback loop designed to expend energy from the battery as needed in order to maintain a certain temperature. Instead of continually supplying a constant current to the heating pad 314, the circuit 210 may supply a current to achieve a desire temperature setting (i.e. idle setting). Once the idle setting is achieved, the circuit 210 may stop sending current from the battery 50, and instead monitor the temperature of the heated pack 100. If the temperature departs from the idle temperature by a certain degree (e.g., 1°, 2°, 3° . . . 10°, etc.) in Celsius or Fahrenheit, the circuit 210 resumes sending current from the battery 50 to the heating pad 314 until the idle temperature is attained again.

In further embodiments, the circuity may be configured to add a boost of temperature in response to command or sensor input. First, the heated pack 100 may have a boost button, that when pressed, causes the heated pack 100 to emit a high level of temperature (e.g., above idle temperature) for a period of time. After the period of time, the heated pack 100 resumes its idle temperature. For a sensory boost, the circuity may be design to sense a temperature drop or sense a certain degree of pressure that indicates a user would like a boost of temperature. The pressure change that triggers the boost may be indicated by application of a certain amount of force by the user. The temperature drop that triggers the boost may be a change in temperature of a certain degree (° C. or ° F.). In some instances, the temperature drop is the change of the external temperature of the heated pack. For example, the heated pack 100 may be turned on and located in a user's jacket pocket. A user may want to warm his/her hands with the heated pack 100, and thus places his/her cold hands in the pocket. The heated pack 100 may sense the presence of the user's hands (i.e. temperature difference or pressure) and initiate a boost in temperature. The circuit 210 may retain the boost in temperature for a set period of time (e.g. 10 seconds, 30 seconds, 1 minute, 2 minute, etc.), and then return to the idle temperature. The length of the boost period may preprogrammed or set by the user.

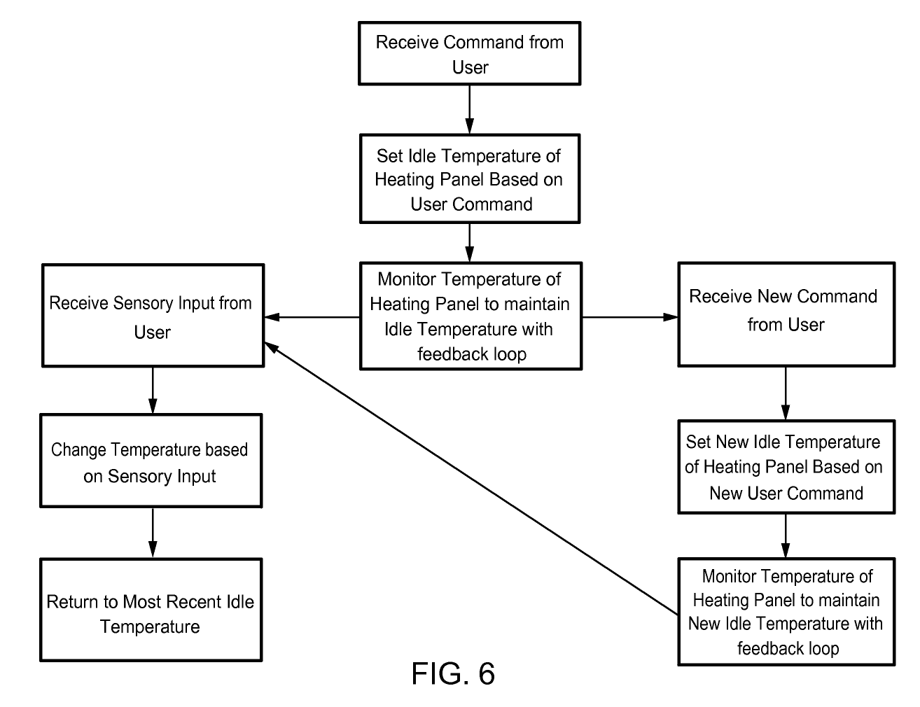

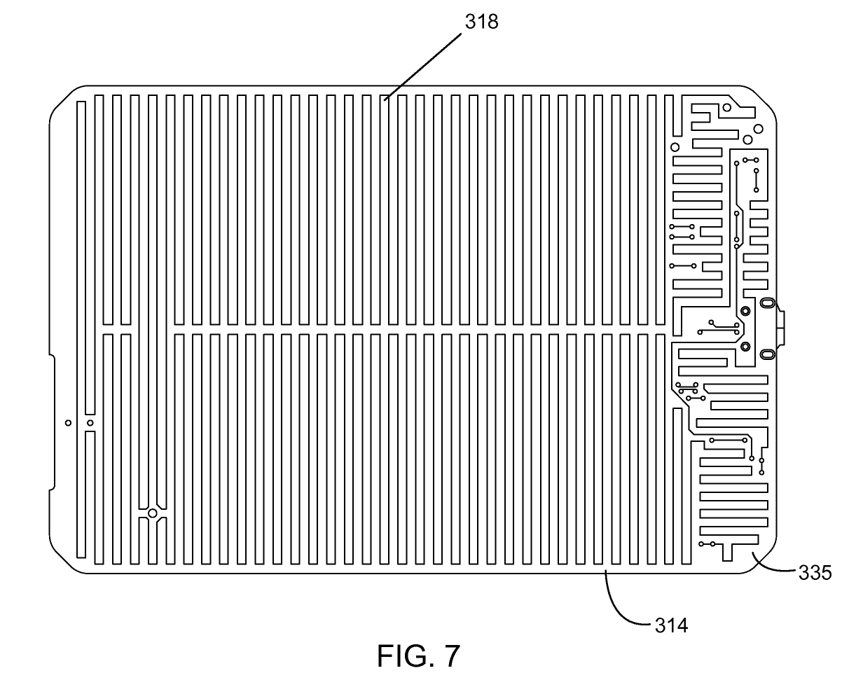

FIG. 6 illustrates an exemplary logic of the circuity 210 for 1) maintaining a temperature while extending the battery life, and 2) providing a higher boost of temperature based on sensory feedback. As shown in FIG. 6, the circuity 210 of the heated pack 100 first receives a command from the user. In response to the command, the circuitry 210 sets an idle temperature of the heating panel 314. The circuity 210 then monitors the temperature of the heating pad to maintain idle temperature using the feedback look. After the initial idle temperature is set, the temperature can be adjusted in two ways. First, the circuity 210 can receive a new user command and set a new idle temperature. Second, the circuity 210 can receive a sensory input (pressure or temperature change) from the user. Upon receiving the sensory input, the circuit 210 provides a boost in temperature. The boost increases the temperature for a period of time, and then the circuity 210 returns the heating pack to its normal temperature.

In certain embodiments, the heated pack 100 includes a battery indicator, a temperature, indicator or both. The temperature indicator may include one or more light emitting diodes (LED) that are associated with circuitry (such as circuit 210 shown in FIG. 4) coupled to the battery. The temperature indicators may show the current temperature setting of the device, e.g. low, medium, high. In certain embodiments, the temperature indicator is a screen that shows the exact temperature in a digital format.

The battery indicator may include light emitting diode (LED) that is associated with circuitry (such as circuit 210 shown in FIGS. 4-5). The battery indicator may be positioned anywhere on the heated pack 100. According to some embodiments, the battery indicator is positioned on the frame so that it is easily visible to a user. FIG. 3B shows a battery indicator 27 positioned in the grasping region 18 of the frame 10. The frame 10 near the battery indicator 27 may include a reflective surface to further enhance the light emitted from the LED. The opening allows light emitted from an LED to be seen therethrough. In one embodiment, the battery indicator emits a light when the battery 14 is charged. The charged-battery light may appear as a single flash, a series of flashes over time, or the light may constantly be emitted while the battery is charged to a certain percentage (e.g. over 25%). Optionally, the battery indicator also emits a light to illustrate that the battery 14 is running low on charge (e.g. below 25%). The low-battery light may appear as a single flash, a series of flashes over time, or constantly emitted light. Preferably, the light emitted to indicate that the battery is properly inserted or connected is different from the light emitted to indicate the battery is low on charge. For example, a green light may indicate the battery is properly inserted, and a red light may indicate the battery needs to be recharged. In addition, the battery indicator may also emit a light to illustrate that the battery 14 is defective, and should be discarded.

The battery 50 of the heated pack 100 may be charged via an external battery charger. The battery charger may be plugged into port 30 (see FIG. 1). According to certain embodiments, the intelligent circuit 210 located in the heated pack 100 monitors the battery charge state and delivers suitable current/voltage to the battery 50 from the external battery charger in order to safely charge the battery 50. Alternatively, the external battery charger may be designed to include its own circuitry that manages delivery of current/voltage from the charger to the battery 50.

Heated packs 100 of the invention are may used as a personal heating device for any number of purposes. Preferably, the heated packs 100 are sized for easy carry on one's person or in one's hand(s). The compact design of heated packs of the invention make them suitable to place in the pockets of articles of clothing (including jackets, t-shirts, pants, shorts, dresses, etcs.) In some instances, the heated packs of the invention may be used to apply heat for therapeutic and medicinal purposes. In other instances, a heated pack may be used to keep an individual warm while participating in outdoor activities (hiking, camping, skiing, etc.).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A personal device for heating, the device comprising:
a frame comprising a base portion and defining a recess;
a heating assembly disposed within the recess and comprising:
a battery;
a control circuit coupled to the battery; and
a heating panel coupled to the control circuit; and
a cover layer coupled to the heating assembly and configured to transition between storing heat and releasing heat in response to energy outputted by the heating assembly.

2. The device of claim 1, wherein the frame comprises a hard polymeric material.

3. The device of claim 1, further comprising a covering that surrounds the frame.

4. The device of claim 1, wherein the cover layer stores heat in response to a first temperature.

5. The device of claim 1, wherein the cover layer releases heat in response to a second temperature.

6. The device of claim 1, wherein the cover layer comprises a phase-change material.

7. The device of claim 6, wherein the phase-change material transitions between solid phase and liquid phase.

8. The device of claim 7, wherein the phase-change material stores heat in the liquid phase.

9. The device of claim 7, wherein the phase-change material releases heat in the solid phase.

10. The device of claim 6, wherein the phase-change material is encapsulated.

11. The device of claim 6, wherein the phase-change material is incorporated into fibers that form the cover layer.

12. The device of claim 1, wherein the control circuit is configured to:
maintain an idle temperature of the heating panel;
receive a sensory input;
change the temperature of the heating panel based on the sensory input; and
return to the idle temperature after a pre-determined period of time.

13. The device of claim 1, wherein the device is sized to held or carried by a user.

14. A personal device for heating, the device comprising
a frame; and
a heating assembly disposed within the frame and comprising a heating panel and a control circuit, wherein the control circuit is configured to;
maintain an idle temperature of the heating panel;
receive a sensory input;
change the temperature of the heating panel based on the sensory input; and
return to the idle temperature after a pre-determined period of time.

15. The device of claim 14, wherein at least one side of the frame is configured to transition between storing heat and releasing heat in response to energy outputted by the heating assembly.

16. The device of claim 15, wherein the at least one side of the frame comprises a phasechanging material.

17. The device of claim 14, wherein the frame comprises a base portion and defines a recess.

18. The device of claim 17, wherein the frame further comprises a cover layer that encloses the recess and transitions between storing heat and releasing heat in response to energy outputted by the heating assembly.

19. The device of claim 14, wherein the sensory input comprises an application of pressure or a change in temperature.

20. The device of claim 16, wherein the base portion comprises a hard polymeric material.

21. The device of claim 14, further comprising a covering that surrounds the frame.

22. The device of claim 14, wherein the device is sized to held or carried by a user.
